(19) United States
(12) Patent Application Publication
(10) Pub. No.: US 2026/0215700 A1
(43) Pub. Date: Jul. 30, 2026

(54) Apparatus for Gait Analysis

(71) Applicant: Günter MICHL, Kiel (DE)

(72) Inventor: Günter MICHL, Kiel (DE)

(21) Appl. No.: 19/150,086

(22) PCT Filed: Feb. 8, 2024
(86) PCT No.: PCT/DE2024/100113

§ 371 Date: Jul. 22, 2025

(30) Foreign Application Priority Data
Feb. 9, 2023 (DE) 10 2023 103 143.0

(51) Int. Cl. A61B 5/11; A61B 5/00; A61B 5/103

(52) U.S. Cl. CPC A61B 5/112; A61B 5/1038; A61B 5/6895

(57) ABSTRACT

The invention relates to an apparatus for analysing the gait of a test subject moving on the device in a movement direction, having a first pressure measurement device designed for receiving one foot of the test subject, a second pressure measurement device disposed next to the first pressure measurement device transversely to the movement direction and designed for receiving another foot of the test subject, and a display and/or evaluation unit connected to the pressure measurement devices, wherein the first pressure measurement device and the second pressure measurement device are each disposed along an individual path oriented in the movement direction of the test subject so as to be movable independently of one another, and at least one control device is provided which is designed at least for performing a movement that brings the pressure measurement devices from a rear position which is opposite the movement direction into a front position which is located in the movement direction.

The invention relates to a device for gait analysis. In particular, the invention relates to a device for pedographic gait analysis that records the kinetic parameters of a subject.

In gait analysis, which records kinetic parameters, a force or pressure measuring plate is used to measure the force or force distribution that the subject's body transfers to the ground when walking or running. The pedographic or pedobarographic images derived from this are used in particular in the rehabilitation of stroke patients, orthopaedic patients, prosthesis wearers and patients with movement disorders, but can also be used for scientific questions, in sports medicine, in sprint and jump training and in the rehabilitation of injuries.

To measure the dynamic force distribution under the sole of the foot during the stance phase of the gait cycle, force measuring plates laid out on the floor are used in particular. These can either be arranged one behind the other to form a track that the subject or patient has to walk along for the gait analysis, or they can be covered by a treadmill belt as part of a treadmill device. Although pressure measuring soles that are inserted into the subject's shoes offer the possibility of direct measurement in the shoe, they are very complex, and there are often fitting problems and difficulties with the transmission cable coming out of the shoe.

In particular, the use of a treadmill device equipped with a force measuring plate is advantageous in that the space requirement and materials input are relatively low compared to running distances formed with force measuring plates and yet a large amount of measurement data can be collected during a running process of any length.

For example, DE 10 2010 003 342 B4 discloses a treadmill device with a belt conveyor system comprising an upper run and a lower run, in which an endless conveyor belt is pulled over a running table, on the underside of which sensors are arranged which can detect local vertical deflections of the running table in various areas of the running table.

However, a disadvantage of the known treadmill devices is that the running track, which is positioned between the force measuring plate and the subject's or patient's foot and also moves over the force measuring plate, distorts the measurement of the dynamic force distribution under the bottom of the foot. The data recorded with a treadmill device and the gait analysis carried out with it are therefore inaccurate compared to the data and the analysis obtained or created using a measuring section consisting of a number of force measuring plates arranged one behind the other.

A device known for gait rehabilitation is known from KR 10-1064891 B1, in which two treadmill belts arranged in parallel are each provided with a pressure sensor arranged on the respective treadmill belt and set up to pick up the left foot or right foot of a patient.

This known device, designed for gait rehabilitation, is unsuitable for gait analysis: firstly, the pressure sensors installed in accordance with the state of the art are only able to indicate the patient's load on the left or right foot and their relationship to each other as a measure of balance, but not to produce a podogram. Rather, the known pressure sensors are used to control the correct positioning of the patient's feet during the movement induced by the device. Secondly, the two treadmill belts and therefore the two pressure sensors are moved in opposite directions by a common timing belt based on a previously set constant time, stride length and speed, so that an unbiased gait analysis is not possible.

Therefore, it is an object of the present invention to improve the known treadmill devices for pedographic gait analysis with their advantage of a small space requirement in such a way that the quality of the data collection and the resulting analysis is comparable to the data that would be recorded by means of a space-intensive running measuring track.

According to the invention, this task is solved by the device with the features of claim 1. The dependent claims describe advantageous embodiments of the invention.

The basic idea of the invention is to utilise the two phases of walking or running to create a space-saving device for pedographic gait analysis. The normal gait sequence consists of the stance phase, which is divided into initial contact with a surface, loading response phase, mid-stance phase, terminal stance phase end and pre-swing phase, during which the subject's foot is in contact with the surface, and the swing phase with initial swing phase, mid-swing phase and terminal swing phase, during which the subject's foot is lifted off the surface and does not touch it. It is understood that during the stance phase, the device must be designed in such a way that the base supporting one foot and having a pressure measuring device can move from a starting position to a position opposite to the running direction in order to simulate a running movement over a normally stationary base. This can be done, for example, by the muscle power of the subjects themselves or by a motor, in particular an electric motor, whereby the base can be designed as a simple plate or as a conveyor belt, for example. It is now essential to the invention that the base, which is moved against the running direction during the stance phase of one foot, is moved in the running direction during the swing phase so that it is available again in the starting position for the initial contact of the foot with the base at the beginning of the stance phase after completion of the swing phase. In principle, therefore, the movement of one pressure measuring device is independent of the movement of the other pressure measuring device in terms of direction, speed and duration of this movement and is determined solely by the walking movement of the subject.

According to the invention, therefore, a device is proposed for analysing the gait of a subject moving in a walking direction on the device, wherein the subject may be a human or an animal, in particular a mammal. The device according to the invention has a first pressure measuring device arranged to receive a foot of the subject, a second pressure measuring device arranged transversely to the direction of movement next to the first pressure measuring device and arranged to receive a further foot of the subject, and a recording and/or evaluation unit connected to the pressure measuring devices. According to the invention, at least two pressure measuring devices are thus provided, each pressure measuring device being designed to accommodate a foot. Accordingly, two pressure measuring devices are generally sufficient for devices used to analyse human gait. However, it is also conceivable that—in particular for using the device to analyse the gait of an animal, for example a dog or a horse—a larger number of pressure measuring devices, in particular four pressure measuring devices, are provided, each pressure measuring device being set up to accommodate one foot.

The first pressure measuring device and the second pressure measuring device are each arranged so as to be movable independently of one another along their own respective path aligned in the direction of movement of the subject, wherein at least one positioning device being provided which is set up at least for executing a movement which moves the pressure measuring devices from a rear position opposite the direction of movement to a front position arranged in the direction of movement.

In other words, the device according to the invention is set up in such a way that, while one foot of a test person enters the swing phase, the other foot remains in the stance phase, whereby the pressure measuring device loaded by the foot remaining in the stance phase executes an autonomous movement independent of the unloaded pressure measuring device, which executes a movement directed in the running direction after unloading. The relieved pressure measuring device is positioned in the running direction in such a way that it is available for a new gait cycle of the foot entering the stance phase from the swing phase to accommodate this foot. In particular, a detection device can be provided for this purpose, which detects the position of the respective foot and causes a corresponding adjustment of the respective pressure measuring device according to the position of the respective foot. A runner will therefore be able to run freely and independently in a straight line on the device according to the invention, whereby the speed and step width are determined by the runner, but not by the device.

This design makes it possible to create a space-saving device for gait analysis that enables direct pedographic gait analysis.

The pressure measuring devices are preferably arranged along parallel tracks. Depending on the subject, however, it may also be possible for the tracks to form an acute angle that is closed in the direction of travel in order to enable the pressure of the subject to be transmitted to the pressure measuring devices as unaltered as possible by the device.

The actuating device is preferably designed as a spring connected to a pressure measuring device. By means of this design, a very simple design of the device can be realised, in which the pressure measuring devices are moved during the walking or running process simulated on the device from the front position seen in the running direction to a rear position arranged against the running direction by means of the subject's muscle power and are automatically pulled into the front position arranged in the running direction by means of the spring when the respective foot is lifted at the beginning of the swing phase.

A further preferred embodiment provides for a (transport) belt carrying a pressure measuring device in each case. In such a case, a first shaft receiving a first partial section of one of the belts arranged in the running direction and/or a second shaft receiving a second partial section of one of the belts arranged against the running direction is particularly preferred. At least the front shaft can have the aforementioned spring, whereby the rear shaft can also have a spring acting against the running direction to achieve a tensioned belt.

Alternatively, or in addition to the design of a spring, the actuating device has at least one motor that moves the pressure measuring devices in the running direction and/or against the running direction. It is particularly preferable that the actuating device is connected to the recording and/or evaluation unit and can receive signals from it to control the motor. In this way, the respective pressure measuring device will be able to detect the lifting of a foot and thus the start of the swing phase, which generates a signal for activating the motor that moves the respective pressure measuring device from the rear position to the (starting) position in front.

Overall, it is also possible to form the belts of both tracks as an endless conveyor belt, whereby, however, there is no rotation, but only a movement of the upper run in the running direction and against the running direction to the extent that the test person can move the respective pressure measuring device from the front position to a rear position, whereupon the device is set up so that it moves the respective pressure measuring device back from the rear position to the front position.

Finally, it is preferably provided that the first pressure measuring device and/or the second pressure measuring device is a pressure measuring plate or a pressure measuring foil. The design as a pressure measuring plate is particularly useful if no belt but a movably arranged plate is provided as the surface to be walked on, whereby the design as a pressure measuring foil is particularly suitable when belts are used.

The advantage of the present invention over a conventional treadmill according to the prior art, in which the movement of the running belt causes the subject's foot to be abruptly pulled backwards against the running direction, so that a non-physiological load is applied to the foot, lies in the fact that when a device designed according to the invention is used, the pressure measuring device, which is movable per se, has no speed at the moment at which the foot strikes the pressure measuring device—this corresponds to the normal condition of human locomotion when walking or running on a solid surface and enables a precise measurement that is directly comparable to static measuring systems. Of course, this also applies if the pressure measuring device is attached to or on a belt.

Whereas with conventional treadmills, the treadmill speed is set by the treadmill belt and the running speed has to be changed manually using a rotary switch or an input button, meaning that a short sprint or an abrupt stop is not possible, a further advantage of the present invention is that by means of the present invention, using acceleration sensors mounted on the feet of the runner, the independent pressure measuring devices can follow each forward and backward movement of the feet of the runner—no matter how fast the movement is and how abruptly it is changed by the runner. For this purpose, the actuating device is specially designed so that the pressure measuring devices are each moved against the running direction at the running speed determined by the acceleration sensors and returned to the starting position in the running direction at half the running speed. This means that the runner's position on the treadmill remains essentially unchanged, but the runner has the feeling of running straight ahead freely and without restriction.

Accordingly, the device designed according to the invention preferably has at least one detection device which is set up to detect the position of the subject's feet during the swing phase and which communicates with the positioning device, the positioning device being set up to track the pressure measuring devices as a function of the position of the respective foot detected by the detection device.

The detection device specifically has a plurality of light barriers set up to detect the position of the subject's feet. Alternatively, or additionally, the detection device has an acceleration sensor set up for attachment to a foot of the subject. The acceleration sensors are used to detect the movement of the respective foot of the subject so that the pressure measuring device can be tracked during the swing phase of a foot—independently of the other pressure measuring device, which is loaded by the foot of the test person in the stance phase—according to the movement of this foot and comes to rest at the beginning of the stance phase at the position at which the foot will touch down on the device during the transition from the swing phase to the stance phase.

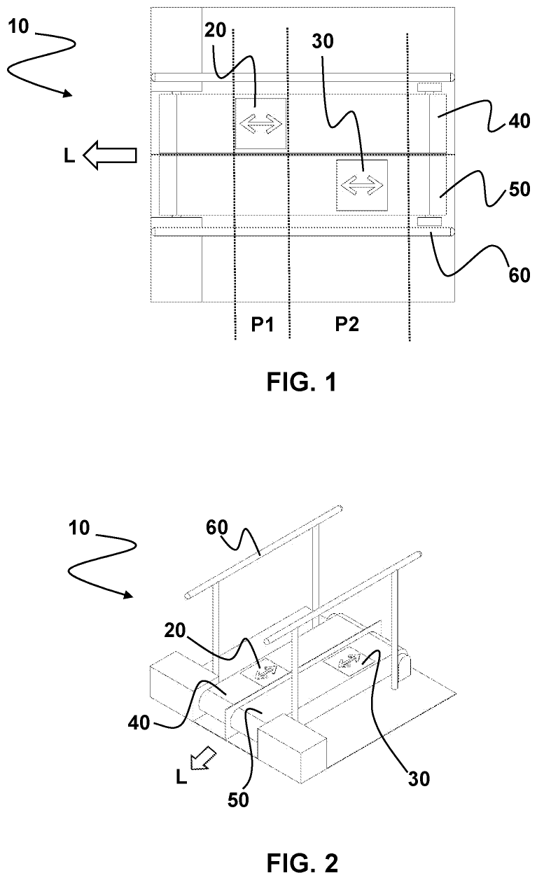

The invention is explained in more detail below with reference to a particularly preferably designed embodiment shown in the accompanying drawings. These show:
FIG. 1 a top view of the particularly preferred embodiment;
FIG. 2 a perspective view of the particularly preferred embodiment shown in FIG. 1.

FIG. 1 and FIG. 2 show a particularly preferably designed example of a device for analysing the gait of a subject moving in a running direction on the device. In particular, the figures show a device 10 set up for analysing the gait of a person. This is similar to a conventional treadmill, but has significant differences to it, which are explained in more detail below:

In particular, it can be seen that the device 10 has a first pressure measuring device 20 set up to receive the subject's right foot and a second pressure measuring device 30 aligned parallel to it and set up to receive the subject's left foot. Both pressure measuring devices 20, 30 are arranged on a separate conveyor belt 40, 50, whereby the conveyor belts 40, 50 each run in a path aligned in the direction of movement L of the test person and can move independently of one another. The pressure measuring devices 20, 30 are arranged on or in the transport belts 40, 50 in such a way that they come into direct contact with the respective foot of the test person and can be moved both in the direction of movement L and against the direction of movement L with the aid of the transport belts 40, 50.

In principle, the pressure measuring devices 20, 30 can each assume two positions P1, P2 along the respective path, namely a front position P1 arranged in the direction of travel L, which represents the starting position, and a rear position P2 opposite the direction of travel L. During simulated walking on the device, the respective pressure measuring device 20, 30 is moved from the front (starting) position P1 to a rear position P2 opposite to the walking direction L during the stance phase of the respective foot by means of the subject's muscle power or motorised by the device 10. If the foot ending the stance phase is lifted from the pressure measuring device 30 arranged in the rear position P 2, the device 10 is equipped with an actuating device (not shown) in such a way that this pressure measuring device 30 is moved from the rear position P2 opposite the running direction L to the front position P1 arranged in the running direction, i.e. to the starting position.

For this purpose, the actuating device can be designed, for example, as a spring connected to a pressure measuring device 20, 30, which is pulled from the rear position P2 to the front position P1 after the load is removed with the subject's foot. For example, such a spring can be provided in a shaft arranged in the running direction L, which receives a first partial section of a respective belt 40, 50 arranged in the running direction L. In addition, a second shaft for receiving the second partial section of the respective belt 40, 50 arranged in the opposite direction to the running direction L would also be advantageous for tightening the respective belt.

According to a particularly advantageous embodiment, however, the actuating device of the device 10 has at least one motor, in particular an electric motor, which moves the transport belts 40, 50 and thus the pressure measuring devices 20, 30 in the running direction L and/or against the running direction L.

The fact that the pressure measuring devices 20, 30 on the upper side of the conveyor belt are only moved in the running direction L and against the running direction L and a lower run—as with conventional conveyor belts—is not absolutely necessary, also ensures a simple (cable-based) connection of the pressure measuring devices 20, 30 with a recording and/or evaluation unit. This can also be connected to the actuating device, for example to signal to the actuating device the start of the swing phase when the foot is lifted from one of the pressure measuring devices 20, 30 and thus give a signal for the movement of the corresponding pressure measuring device 20, 30 from the rear position to the front position.

The pressure measuring devices 20, 30 can be designed as a pressure measuring plate or as a pressure measuring foil, whereby the device 10 can have further devices for gait analysis, for example cameras, or for supporting the test person on the device 10, for example a handrail 60 delimiting the longitudinal sides of the device 10.

1. An apparatus for analysing a gait of a subject moving on the apparatus in a walking direction, comprising
a first pressure measuring device arranged to receive a foot of the subject,
a second pressure measuring device arranged transversely to the direction of movement next to the first pressure measuring device and arranged to receive a further foot of the subject, and
a recording and/or evaluation unit connected to the pressure measuring devices, wherein
the first pressure measuring device and the second pressure measuring device are each arranged so as to be movable independently of one another along their own respective path aligned in the direction of movement of the subject, and
at least one actuating device is provided which is set up at least for executing a movement which moves the pressure measuring devices from a rear position opposite the direction of travel to a front position arranged in the direction of travel.

2. The apparatus according to claim 1, wherein the paths are aligned parallel to one another.

3. The apparatus according to claim 1, wherein the actuating device in each case is designed as a spring connected to a pressure measuring device.

4. The apparatus according to claim 1, further comprising a respective belt each carrying a pressure measuring device.

5. The apparatus according to claim 4, further comprising a first shaft receiving a first partial section of one of the belts arranged in the direction of movement and/or a second shaft receiving a second partial section of one of the belts arranged counter to the direction of movement.

6. The apparatus according to claim 1, wherein the actuating device has at least one motor moving the pressure measuring devices in direction of movement and/or against the direction of movement.

7. The apparatus according to claim 1, wherein the first pressure measuring device and/or the second pressure measuring device is a pressure measuring plate or a pressure measuring foil.

8. The apparatus according to claim 1, further comprising at least one detection device which is set up to detect the position of each foot of the subject during a swing phase and communicates with the actuating device, the actuating device being set up to track the pressure measuring devices as a function of the position of the respective foot detected by the detection device.

9. The apparatus according to claim 8, wherein the detection device has a plurality of light barriers set up to detect the position of the subject's foot.

10. The apparatus according to claim 8, wherein the detection device comprises an acceleration sensor adapted to be attached to the foot of the subject.
